(19) United States
(12) Patent Application Publication
(10) Pub. No.: US 2021/0292680 A1
(43) Pub. Date: Sep. 23, 2021

(54) MULTIFUNCTIONAL AROMATIC ALCOHOLS FOR PERSONAL CARE, HOME CARE AND INDUSTRIAL AND INSTITUTIONAL COMPOSITIONS

(71) Applicant: EMERALD KALAMA CHEMICAL, LLC, Vancouver, WA (US)

(72) Inventors: Bradley Les FARRELL, Vancouver, WA (US); Kelly Reed PIPPINE, Vancouver, WA (US); Stephen Finley FOSTER, Ridgefield, WA (US); Rosanna Williamson STOKES, Charlotte, NC (US); Amber YARNELL, Vancouver, WA (US); Natalie Irene KEEHAN, Longview, WA (US); Sarah L. STROTHER, Woodland, WA (US)

(21) Appl. No.: 17/325,323

(22) Filed: May 20, 2021

Related U.S. Application Data
(63) Continuation-in-part of application No. PCT/US2019/062422, filed on Nov. 20, 2019.
(60) Provisional application No. 62/769,949, filed on Nov. 20, 2018.

(51) Int. Cl. C11D 3/00; A61K 8/34; A61Q 1/14; A61Q 19/00; C11D 11/00; C11D 3/20; C11D 3/43; C11D 3/04; C11D 3/33; C11D 1/28; C11D 1/83

(52) U.S. Cl. CPC C11D 3/0026; C11D 1/22; A61Q 1/14; A61Q 19/00; A61Q 19/007; C11D 11/0017; C11D 3/2034; C11D 3/43; C11D 3/2086; C11D 3/2065; C11D 3/2044; C11D 3/046; C11D 3/33; C11D 3/044; C11D 1/28; C11D 1/83; A61K 8/345

(57) ABSTRACT

Single ingredient, multifunctional additives for use in a wide variety of products comprise aromatic alcohols including without limitation 3-phenylpropanol, 2-phenylethanol, 2-methyl-3-phenylpropanol, methyl benzene ethanol, methyl benzene propanol, or 4-phenyl-1-butanol, or mixtures thereof. The multifunctional aromatic alcohols provide unexpected improvements in processing and in end use product properties of a wide variety of products and are useful at a wide range of pH.

### FIELD OF THE INVENTION

The invention is directed to single ingredient, multifunctional additives for use in a wide variety of products to provide improvements in manufacturing/processing and end use properties of products and/or reduce or eliminate the need for single function components in product formulations. The invention is also directed to a wide variety of products, including without limitation industrial, institutional, home care (household), laundry, detergent, cosmetic, and personal care compositions, containing the multifunctional additives of the invention.

### BACKGROUND OF THE INVENTION

As ingredients in personal care, cosmetic, home care (household), laundry, detergent, industrial, and institutional products have persistently attracted regulatory, industry, and consumer attention in recent years, the need for constant innovation in formulating and manufacturing such products continues to grow. Many ingredients, including for example antimicrobials, preservatives, solvents, and fragrances have received government and regulatory attention, requiring concentration restrictions and even prohibitions on use in certain applications and prompting manufacturers to explore alternatives that are environmentally friendly and/or safer to handle and use. In addition, manufacturers are looking to simplify ingredient lists for cost savings, improved inventory and logistics, as well as address consumer needs for transparency.

As a consequence, formulators are seeking chemical ingredients which can add multiple performance characteristics, i.e., functions, to formulations and reduce or eliminate the need for a multitude of other single function ingredients. In short, formulators are focused on selecting materials that serve to improve the performance of a number of processing and end use characteristics or properties of a formulation using a single ingredient that has multiple functions. As one example, preservative boosters like 1,2-hexanediol and caprylyl glycol enjoy increasing use over more traditional preservatives as they not only improve antimicrobial properties but also improve emollience in certain applications. As well, many polyethylene glycol derivatives are used both as rheology modifiers and emulsifiers.

As another example, EP 1 206 933 A1 relates to compositions containing caprylyl glycol or an analog thereof as a humectant and an emollient and as a compound to increase the activity of a preservative agent. It further relates to the use of such compositions in topical formulations, in particular in cosmetic formulations. Caprylyl glycol is stated to be devoid of undesired effects to the skin and in particular to atopic skin, noting its current use as humectant or emollient.

Similarly, U.S. Pat. No. 6,500,411, B2 teaches the use of a particulate additive composition that when added as a component in personal care and cosmetic formulations, can simultaneously provide one or more of the following functions: i) improve the efficacy of sunscreen actives used as ultraviolet radiation (UVR) filters, ii) thicken hydrophilic solvents such as water, glycols, glycerin, and alcohols or mixtures of these solvents, iii) emulsify and stabilize oil droplets in oil-in-water (O/W) emulsions, and iv) function as an antioxidant.

U.S. Pat. No. 6,541,443 B1 discloses the use of a multifunctional detergent material useful in a laundry or cleaning product containing condensed phosphate, a silicate, and optionally other organic materials. The composition is described as providing multiple functions for laundry and cleaning products in a “single” ingredient, including the functions of a builder, a filler, and an alkaline source, although more than one chemical compound is used to prepare the “single ingredient” material.

It has been discovered that certain aromatic alcohols, when used as a single ingredient in a formulation, demonstrate multiple functions and unexpectedly improve in process and end use properties of certain compositions. In particular, it has been found that certain aromatic alcohols serve to: i) modify low- and high-shear rheology (viscosity) of oil-in-water emulsions and aqueous surfactant solutions; ii) reduce the stability of process foam generated by surfactant-based formulations during manufacturing; iii) increase the stain removal efficacy of laundry products; iv) act as a degreaser in personal care and household cleaning formulations; and v) solubilize surfactants, polymers, gums, long-chain glycols, and other hard-to-dissolve solids in aqueous solutions or formulations. The aromatic alcohols of the invention have the structure set forth in Formula I below, where R=an aliphatic saturated alkyl group consisting of 2-4 carbons. This includes without limitation 3-phenylpropanol, 2-phenylethanol, 2-methyl-3-phenylpropanol, methyl-benzene ethanol, methyl-benzene propanol, and 4-phenyl-1-butanol. Other aromatic alcohols of Formula I will be known to one skilled in the art.

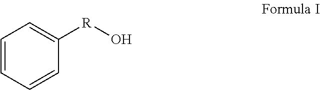

The fragrance attributes of aromatic alcohols are known to one skilled in the art. As these compounds are low molecular weight and carry a fairly low vapor pressure at ambient temperature, they have been used as fragrances, fragrance precursors, and odor-masking agents. By way of example, EP 2 127 632 A1 relates to perfume compositions which are free of lower alcohols such as ethanol. To obtain a fragrance mixture with a combination of perfume oils, use of aromatic substances is disclosed, including aromatic alcohols such as benzylic alcohol, cis-3-hexenol, phenyl ethyl alcohol, anysil alcohol, isoamyl alcohol, 4-hexen-1-ol, phenoxyethanol; aromatic substances such as ethyl vanilline, vanilline, heliotropine, helional, coumarin, ethyl maltol, ethyl acetate, ethyl acetoacetate, 2-methyl pyrazine, linalool oxide; non-odour substances such as phenoxypropanediol, trimethyl-1,3-pentanediol, chlorphenesin, ethylhexyl glycerin, caprylyl glycol, glyceryl caprylate; and mixtures thereof.

Certain aromatic alcohols have also been used as antimicrobials, usually in combination with other antimicrobials. As one example, benzyl alcohol is a known preservative compound. By way of illustration, U.S. Pat. No. 6,120,758 discloses use of benzyl alcohol in combination with phenoxy ethanol and phenoxy propanol and glycerol ethers as effective stabilizing compositions for cosmetic products. In addition, U.S. Pat. Nos. 7,537,776 and 8,501,206 disclose benzyl alcohol in combination with a sorbic acid salt or sodium benzoate and/or phenoxyethanol for use as a preservative concentrate for cosmetics. As other examples, WO 2016 084078 discloses blends of 3-phenylpropanol in amounts ranging from 2 up to 30 wt. % with an organic acid (selected from the group consisting of dehydroacetic acid (DHA), benzoic acid or mixtures thereof), a diol or triol (selected from the group consisting of a 1,2-diol 1,3-diol or mixtures thereof), and the like. WO 2014 135650 discloses antimicrobial efficacy for blends of 3-phenylpropanol mixed with acetophenone. Similarly, WO 2016 164555 discloses antimicrobial efficacy for blends of 3-phenylpropanol and propylene carbonate.

Some aromatic alcohols may be present in formulations as one component of certain multicomponent additives claimed to be and advertised commercially to be “multifunctional”; however, they are not used alone, but rather in combination with other components each of which contribute to the “multifunction” of the additive.

Notwithstanding the above-noted prior uses of aromatic alcohols, the use of the aromatic alcohols of the invention as single ingredients having multiple functions to eliminate or reduce the need for other ingredients in product formulations has not been explored outside of their potential antimicrobial or fragrance properties. The unexpected added improvements in processing and end use properties noted above provide alternatives for formulating products that are easier to manufacture and have fewer environmental and health and safety issues. As one example only, it has been discovered that certain aromatic alcohols of the invention, when added to surfactant formulations for use in home care and personal care applications, are able to reduce foam occurring during the manufacturing process by overhead addition of the alcohols with stirring. This unexpected property is especially crucial, as regulators are aiming to limit silicones in personal care formulations due to bioaccumulation and ecotoxicity. It has also been found that use of these aromatic alcohols during the manufacturing process do not alter the characteristics of the final products, including their foaming capabilities.

Some aromatic alcohols have been utilized in certain personal care applications to reduce foaming. U.S. Pat. No. 10,071,035 B2 discloses the use of benzyl alcohol 0.1 to 0.7 wt. % as both a defoaming agent and an antimicrobial agent in an oral care composition comprising a surfactant. Benzyl alcohol's defoaming activity is stated to be improved by the use of certain flavoring agents in addition to benzyl alcohol. A method of manufacturing an oral care composition is also disclosed, wherein a surfactant is combined with the oral care base composition at a point no later than the addition of the defoaming agent.

The aromatic alcohols of the invention have not heretofore been used as viscosity modifiers, process foam reducers, stain removal additives, degreasers, and solubilizing agents for surfactants, polymers, gums, and long-chain glycols. The unexpected improvements in processing and end use properties coupled with antimicrobial and low or no odor effects provide a viable alternative to reduce the ingredient load of a wide variety of products, including without limitation home care, household, laundry, detergent, personal care, cosmetic, industrial, or institutional products by using a multifunctional ingredient in place of several single function ingredients.

It is an object of the invention to provide a single ingredient, multifunctional additive for use in a wide variety of personal care, cosmetic, laundry, detergent, household, home care, industrial, and institutional compositions to provide improved rheology, process foam reduction, stain removal, degreasing, and solubilization properties when added to the composition as a single ingredient.

It is another object of the invention to provide methods for modifying rheology, reducing manufacturing process temperature and time to produce an end use product, reducing and destabilizing process foam, solubilizing components, and improving stain removal properties for a wide variety of end use compositions.

Another object of the invention is to provide end use compositions comprising the aromatic alcohols of the invention.

Yet another object of the invention is to reduce the number of single function components in an end use composition by utilizing a single ingredient, multifunctional additive to eliminate or reduce the need for separate rheology modifiers, fragrances, process foam control agents, stain removers, degreasers, and solubilizing or stabilizing components.

Other objects of the invention will be evident to one skilled in the art based upon the disclosure herein.

### SUMMARY OF THE INVENTION

The invention is directed to use of single ingredients for use as multifunctional additives in a wide variety of end use products, including without limitation personal care, cosmetic, household, home care, laundry, detergent, industrial, or institutional products to eliminate or reduce the need for separate single function ingredients. The invention is also directed to methods for improving the manufacturing process for end use products by use of the multifunctional ingredients of the invention to modify rheology, reduce manufacturing temperatures and time, reduce or destabilize foam in process, and solubilize components. The invention is also directed to end use products comprising the multifunctional ingredients of the invention to improve properties of end use compositions, including without limitation viscosity stability, stain removal, and degreasing.

In one embodiment, the invention is directed to a multifunctional ingredient for use in a variety of end use products comprising an aromatic alcohol having the formula:

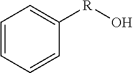

wherein R=an aliphatic saturated alkyl group consisting of 2-4 carbons.

In a second embodiment, the invention is directed to a method for modifying the rheology of oil-in-water emulsions and aqueous surfactant compositions comprising the step of adding an aromatic alcohol to the composition.

In a third embodiment, the invention is directed to methods for reducing manufacturing process temperatures, process time, and reducing or destabilizing foam in the process environment comprising adding an aromatic alcohol to the composition during processing.

In a fourth embodiment, the invention is directed to a method for solubilizing polymers, gums, long chain glycols, and surfactants in certain home and personal care formulations, among other formulations, comprising the step of adding an aromatic alcohol to the formulations.

In a fifth embodiment, the invention is directed to end use compositions comprising the aromatic alcohols of the invention, including without limitation, laundry detergents, household products, home care products, personal care compositions, cosmetic formulations, industrial, and institutional compositions to improve properties such as viscosity stability, stain removal, and degreasing.

In a sixth embodiment, the invention is directed to a method for eliminating single function ingredients in an end use product, by addition of the aromatic alcohols of the invention.

Other embodiments will be known to one skilled in the art based upon the disclosure herein.

### BRIEF DESCRIPTION OF THE DRAWINGS

FIG. 1 is a shear ramp curve of skin lotions with an aromatic alcohol (R=3 carbons) at various levels and without an aromatic alcohol (control) showing viscosity responses (Pa·s) across the tested shear (1/s) band.

FIG. 2 shows relative ratings for integrity of shape of Suave Essentials® with and without increasing concentrations of an aromatic alcohol (R=3 carbons).

FIG. 3 is a shear ramp curve for hand dish washing soap dosed with varying amounts of an aromatic alcohol (R=4).

FIG. 4 shows foam loss (based on % of initial foam volume as determined by modified ASTM E2407-04 methodology) showing the effects on foam loss of using 0.4 wt. % aromatic alcohol defoamers (R=3, R=4) in sodium laureth sulfate (SLES) and BioSoft® N1-7 surfactant solutions.

FIG. 5 shows foam loss using different commercial products as surfactant sources and demonstrates that the addition of 0.5 wt. % aromatic alcohol (R=3) does not reduce the foam stability of the finished product.

FIG. 6 shows laundry stain removal results for a variety of stains using 0.5 wt. % of an aromatic alcohol where R=3.

FIG. 7 shows the results of greasy stain removal test using Fabuloso® all-purpose cleaner and 0.5 wt. % aromatic alcohol (R=3), wherein the upper portion of the tiles was cleaned with Fabuloso® containing 0.5 wt. % aromatic alcohol (R=3) and the bottom portion of the tiles was the control cleaned with Fabuloso® alone.

FIG. 8 shows the results of greasy stain removal test using Fabuloso® all-purpose cleaner and 0.5 wt. % aromatic alcohol (R=4), wherein the upper portion of the tiles was cleaned with Fabuloso® alone, while the lower portion of the tiles was cleaned with Fabuloso® containing 0.5 wt. % aromatic alcohol (R=4).

FIG. 9 Shows makeup removal results for Rimmel London® Lasting Finish® lipstick (top panels) and Maybelline® Superstay™ foundation (bottom panels) when using Garnier SkinActive® micellar water with (Column X) and without (Column C) 1.0 wt. % aromatic alcohol (R=3).

FIG. 10 shows stability of a general purpose cleaner (having a pH of 10 (a), a pH of 7 (b) and a pH of 5 (c)) containing hydroxyethyl cellulose or xanthan gum, after storage at room temperature for one week, with (right) and without (left) 0.5 wt:% aromatic alcohol (R=3).

FIG. 11 compares the homogeneity of a general purpose cleaner (pH 7) in the absence of hydroxyethyl cellulose or xanthan gum, but containing coco fatty alcohol sulfate surfactant, with (right) and without (left) incorporation of an aromatic alcohol (R=3) at 0.5 wt. % and shows that the sample without the aromatic alcohol contained precipitated surfactant while the sample containing the aromatic alcohol remained homogeneous.

The testing reflected in the figures is more fully explained in the description of the invention and the examples herein.

### DETAILED DESCRIPTION OF THE INVENTION

The invention is directed to single ingredient, multifunctional additives, comprising aromatic alcohols, for use in a wide variety of products, including without limitation personal care, cosmetic, household, home care, laundry, detergent, industrial, or institutional products to eliminate or reduce the need for separate single function ingredients. The invention is also directed to methods for improving the manufacturing process for products by use of the multifunctional ingredients of the invention to modify rheology (viscosity), reduce manufacturing temperatures and time, reduce or destabilize foam in process, and solubilize components. The invention is also directed to end use products comprising the multifunctional ingredients of the invention to improve the properties of end use compositions, including without limitation viscosity stability, stain removal, and degreasing.

For purposes of the invention, “ingredient”, “additive”, “component”, and “compound” are used interchangeably to refer to single components, unless otherwise specified.

“Composition” and “formulation” shall mean an end use product and all of its ingredients. “Composition”, “formulation”, and “product” are used interchangeably herein.

“Process” shall mean and include a production or manufacturing process for producing or manufacturing a product. “Process”, “production” and “manufacturing”, and their derivative forms, are used interchangeably herein.

A “home care” or “household” product shall mean and include without limitation products purchased by individuals for use in and around the home for cleaning and other tasks related to the care of the home. “Home care” and “household” are used interchangeably herein.

An “industrial product” shall mean and include without limitation a product bought and used for industry or business purposes, distinct from consumable products bought and used for consumption and satisfaction of individual or home needs such as home care, household, or personal care products.

An “institutional product” shall mean and include without limitation a product that is designed to be used primarily in the maintenance or operation of an establishment that manufactures, transports, or sells goods or commodities, or provides services for profit, or is engaged in the non-profit promotion of a particular public, educational, or charitable cause, such as schools, universities, hospitals, charities, clubs, and similar organizations that buy products for use in producing their own goods or providing services.

A “personal care product” shall mean a product purchased, consumed and used by an individual to satisfy personal individual needs and includes a wide variety of products, including without limitation beauty products, hair products, skin products, dental products, cosmetics, toiletries, fragrances, and the like.

“Aromatic alcohol”, with respect to the invention, shall mean and include those alcohols having the formula:

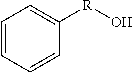

wherein R=an aliphatic saturated alkyl group consisting of 2-4 carbons.

“Multifunctional” when used to refer to the aromatic alcohols of the invention means a single aromatic alcohol that provides multiple functions when used in the manufacture of a product or in an end use product.

The aromatic alcohols of the invention include without limitation those alcohols having an aliphatic saturated alkyl group consisting of 2-4 carbon atoms (R=2-4). Suitable alcohols include: 3-phenylpropanol (R=3), 2-phenylethanol (R=2), 2-methyl-3-phenylpropanol (R=4), methyl benzene ethanol (R=3), methyl benzene propanol (R=4), 4-phenyl-1-butanol (R=4), or mixtures thereof. The aromatic alcohols of the invention are effective used alone and are not required to be mixed with other aromatic alcohols, although they may be in some applications. Other aromatic alcohols having an aliphatic saturated alkyl group consisting of 2-4 carbon atoms will be known to one skilled in the art. Based on experimentation to date, the aromatic alcohols of the invention all achieve unexpected improvements in rheology, reduction in manufacturing temperatures and time, reducing or destabilizing foam in process, and solubilizing components, as well as improvements in viscosity stability, degreasing, and stain removal of end use products.

The aromatic alcohols of the invention are added directly to an end use composition (product) prior to processing or may be added during processing of the product.

The aromatic alcohols of the invention are added to the composition in amounts up to 2.0 wt. %, preferably between 0.1-0.5 wt. %, based upon the total weight of the composition. Amounts used may vary depending on the application in which the aromatic alcohol is being used. By way of example only, suitable amounts to modify low and high shear rheology of oil-in-water emulsions and aqueous surfactant containing compositions is between 0.1 and 0.5 wt. %, based on the total weight of the emulsion or composition. Suitable amounts to reduce manufacturing temperature and or time to manufacture a formulation preferably range from 0.2 to 1.0 wt. %. Suitable amounts for destabilizing foam in a process environment are around 0.2 to 0.5 wt. %. Suitable amounts for improving stain removal of a laundry detergent are about 0.5 wt. %. Other ranges may be used as needed depending on other components included within the composition and are within the scope of the invention.

As described further herein, the invention is directed to use of the multifunctional additives in end use compositions to improve processing and end use properties of a composition. Aside from their known fragrance and/or antimicrobial properties, the aromatic alcohols of the invention unexpectedly improve rheology, reduce or destabilize process foam, solubilize polymers, gums, long chain glycols, and surfactants, and improve end use properties such as viscosity stability, stain removal, and degreasing. Heretofore, none of the aromatic alcohols of the invention were known or reasonably expected to achieve such results when used as a single component in a formulation.

As general observations in production/processing, it has been found that use of the aromatic alcohols of the invention can increase the viscosity of products and reduce splashing or spills in the process environment, resulting in a cleaner and safer process environment and reduced equipment maintenance. In addition, the use of the aromatic alcohols allow for cold processing and reduced heat in production, which is beneficial for formulations comprising heat-sensitive ingredients and volatile materials, including but not limited to solvents, enzymes, prebiotics and probiotics, polymers, dyes, fragrances, preservatives, and natural extracts. Reduced heat puts less stress on manufacturing equipment making the process cleaner and safer.

In many instances, use of the aromatic alcohols of the invention will also reduce or eliminate the need for other components in the process. Use of the multifunctional aromatic alcohols of the invention reduces inventory, number of tanks used in the process, and the number of ingredients required to be identified on products.

Solubilizing properties of the aromatic alcohols allow for preparation of pre-blend (pre-process) solutions or concentrates of hard to solubilize components and reduce solids handling during production, which is difficult and often messy.

As is clear, the addition of aromatic alcohols provide advantages to the production process as they conserve energy, are more “green” or efficient, save time, and increase production/throughput, all of which reduce the costs of production.

As additives in laundry detergents or other degreasing cleaners, the aromatic alcohols of the invention may provide viable options to the use of enzymes for greasy stain removal. Use of enzymes, while popular, are constrained by pH as enzymes require pH between about 6.5 and 8.5, whereas the aromatic alcohols of the invention have been shown to enhance cleaning performance over a wider range of pH, including neutral and acidic pH compositions that cannot use enzymes.

Similarly, use of the aromatic alcohols of the invention in personal care cleansers not only enhances cleaning performance of the product, but can also improve the consumer experience in product attributes such as richness and skin feel.

The multiple functions of the aromatic alcohol additives of the invention and their use in end use applications is further described by the following non-limiting examples.

### EXAMPLES

Test Methodologies. Methodologies used in the examples are described below:

Shear Ramp oil-in-water emulsion: A 25 mm parallel plate geometry was used on an AR2000ex rheometer at 25° C. A gap of 600 μm was used. The shear was ramped from 0 s⁻¹ to 100 s⁻¹ over one minute. Viscosity readings are expressed in Pa·s.

Shear Ramp aqueous solution: The 40 mm cone and plate geometry was used on an AR2000ex rheometer at 25° C. A gap of 29 μm was used. The shear was ramped from 0 s⁻¹ to 2000 s⁻¹ over six minutes. Shear rate is expressed in s⁻¹ or 1/s. Viscosity readings are expressed in Pa·s.

Integrity of shape: In a petri dish, a trained panelist dispensed the product in a spiral shape using a nickel size circle, filling it from the edge to the center. A series of trained panelists then evaluated the product for its ability to maintain shape on a scale from 0 to 100, with 100 representing excellent shape retention.

Laundry stain removal: The AISE stain set was used and tested by an external lab following the AISE Laundry Detergent Testing Guidelines.

Greasy Stain Removal: Testing followed HCPA Cleaning Compendium method DCC-17.

Defoaming evaluation: Process defoaming was tested using a modified ASTM method (E2407-04 (2015) Standard Test Method for Effectiveness of Defoaming Agents). 250 mL of a 2.0 wt. % surfactant solution was blended at the highest speed setting for 30 seconds, then let sit for 3 minutes. The initial foam height is measured, and then 0.4 wt. % defoamer is dropped onto the surface of the foam. The blender is turned on low speed and the solution is mixed for 1 minute. Foam height is re-measured after an additional five minutes of sitting.

Final product foam stability was tested using Blender Foam Volume/Drainage method published by Henkel Corp. 1981.

Time to dissolve: Aromatic alcohol, 1% by weight, was added to deionized water to make 500 mL of solution. Stirring was started at 300 rpm. Test ingredient was added to both a 500 mL control deionized water and the 500 mL aromatic alcohol solution as a stopwatch timer was started. The timer was stopped when solution was completely clear and/or no particles of the test ingredient were visible in either sample. This is noted as the “dissolution time”, or time to dissolve.

Makeup Removal: A new makeup applicator sponge was used to apply makeup samples—0.25 g for lipstick and 0.50 g for foundation—to white card stock, spreading evenly in a stripe across the board. Makeup was allowed to dry for 30 minutes, then excess was removed gently with a lint-free lab wipe. Test and control solutions were prepared and applied to a dry cellulose-based flushable wipe using solution mass of three times the wipe mass. The moistened wipe was wrapped around a new polyethylene foam block and inserted into the scrub tester and additional mass was placed on the block holder for a total holder mass of 700 g. Sample was scrubbed using a pneumatic scrub tester set at 6 seconds per cycle for one-half of a cycle. Test boards were dried overnight and analyzed using spectrophotometer reflectance “L” values according to Equation 1.

$$\% Cleaning efficiency = \frac{C - S}{U - S} \times 100 \qquad ( Equation 1 )$$

Where:
C=Reflectance value of cleaned panel
S=Reflectance value of soiled panel
U=Reflectance value of unsoiled panel

### Example 1. Viscosity Modifier

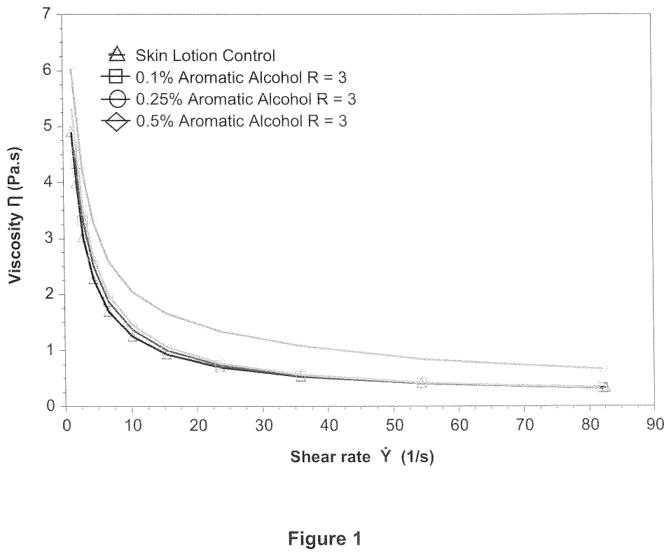

R=3 Aromatic Alcohol. An R=3 aromatic alcohol, i.e., 3-phenylpropanol, was tested in a commercial oil-in-water emulsion, i.e., Vanicream® skin lotion, at 0.1 wt. %, 0.25 wt. % and 0.5 wt. % addition and compared to a control (without aromatic alcohol). It was found that the aromatic alcohol (R=3 carbons, 3-phenylpropanol) had a profound effect on low (≤15 s⁻¹) and high (>15 s⁻¹) shear rheology at concentrations up 0.5 wt. % of aromatic alcohol, based on the total weight of the emulsion. The low shear (at 10 s⁻¹) viscosity was increased 68% over the control, while high shear viscosity (at 70 s⁻¹) increased roughly 100% (FIG. 1). The increase in high and low shear viscosity achieved desirable rheological traits such as richness, creaminess, and enhanced texture and skin feel in this end use personal care product with the addition of the aromatic alcohol. This unexpected result allows personal care and home care formulators, among others, to reduce or eliminate use of separate rheology modifiers to manipulate the viscosity of the final product.

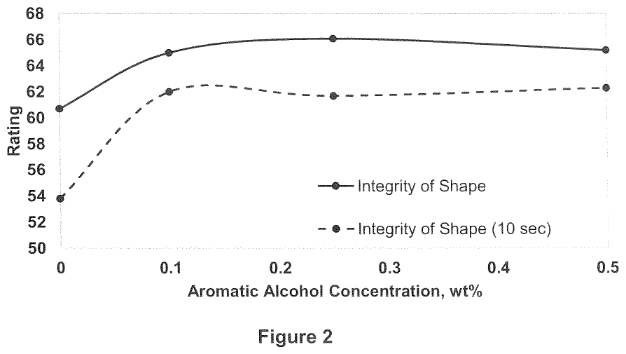

An example of enhanced rheological traits was also demonstrated by integrity of shape ratings of a formula as tested by a trained sensory panel on Suave Essentials® Conditioner (FIG. 2). Relative ratings for integrity of shape were determined for samples without (0 wt. %) and with increasing concentrations (0.1, 0.2, 0.3, 0.4 and 0.5 wt. %, based on the total weight of product) of an aromatic alcohol (R=3)(3-phenylpropanol) by a trained panel. An increased or higher rating of integrity of shape heightened the consumer's perceived luxuriousness or richness of the formulation.

The use of an aromatic alcohol (R=3 carbons, 3-phenylpropanol) in a hand dish wash aqueous surfactant solution demonstrated an increase in viscosity when used in amounts up to 1.0 wt. % as compared to a control subjected to the same shear. At 0.5 wt. %, the low shear viscosity increased roughly 36% over the control, while high shear viscosity increased roughly 43%. (Results not shown.)

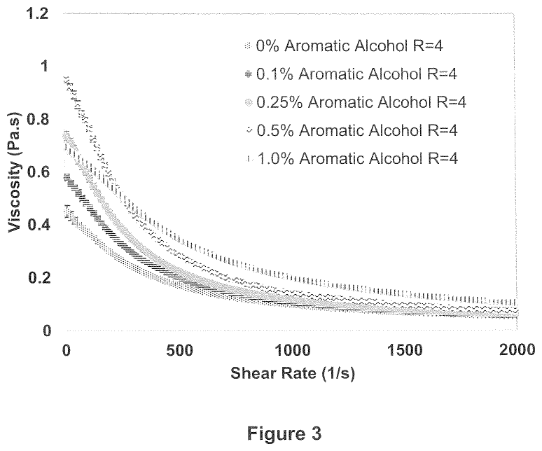

R=4 Aromatic Alcohols. Similarly, viscosity modifying behavior is observed in a hand dish wash aqueous surfactant solution using an aromatic alcohol, where R=4 carbons (2-methyl-3-phenylpropanol). FIG. 3 shows that with use of up to 0.5 wt. % aromatic alcohol, there was a consistent increase in viscosity for samples containing an aromatic alcohol across all shear rates, most notably at low shear rates (≤500 s⁻¹). For 0.5 wt. % aromatic alcohol, the low shear viscosity increased 110% over the control, while high shear viscosity increased approximately 37%. At 1.0 wt. % aromatic alcohol, the viscosity profile changed, yielding increases in low and high shear viscosity of 56% and 94%, respectively.

### Example 2. Defoaming Function

Foam suppression can be a vital part of the manufacturing process, preventing spills and mechanical failures while ensuring the appropriate dosing of ingredients. Aromatic alcohols unexpectedly were shown to be effective agents in dealing with process foam buildup without significantly compromising the foam stability of the final product.

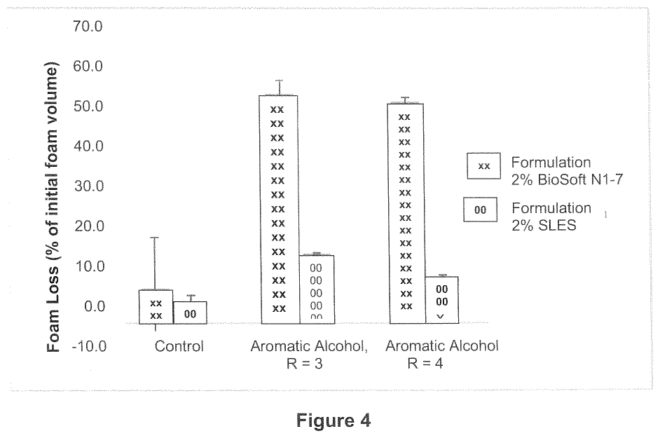

Process defoaming was tested using a modified ASTM method (E2407-04 (2015) Standard Test Method for Effectiveness of Defoaming Agents) to assess surfactants and determine if there was a relationship between foam destabilization and surfactant type. Two different surfactant solutions were tested. 2.0 wt. % surfactant solutions were prepared as controls using BioSoft® N1-7 and SLES. Thereafter, 0.4 wt. % aromatic alcohols (R=3 and R=4) were added to each surfactant solution for comparison to the controls. After agitation to generate foam, the percent of initial foam volume loss was far less for the control (without aromatic alcohols). The results showed that aromatic alcohols (R=3 or 4) (3-phenylpropanol or 2-methyl-3-phenylpropanol) significantly destabilized foam of a nonionic linear alcohol ethoxylate surfactant (BioSoft® N1-7) (FIG. 4). There was a very mild defoaming effect on the high-foaming surfactant, SLES, suggesting that the amount of foam loss may vary with surfactant type when the amount of aromatic alcohol is constant.

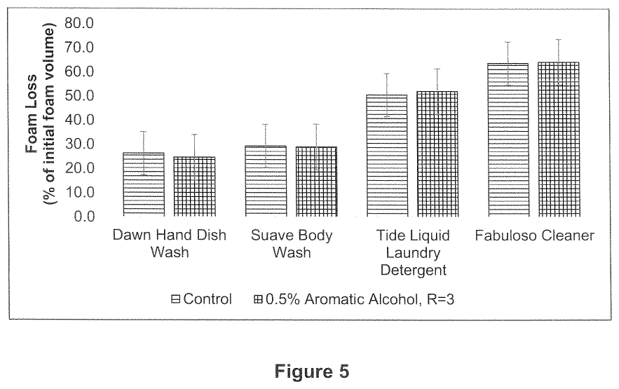

In the foam loss evaluation above, foam destabilization occurred at the time of addition (with mixing) of the aromatic alcohol. Further testing was performed to determine if the foaming ability of final products would be negatively impacted if aromatic alcohols were added to the formulations during the production process. A variety of commercial products were selected for testing, and 0.5 wt. % of aromatic alcohol (R=3, 3-phenylpropanol) was added to each. FIG. 5 shows foam loss (% of initial foam volume) for a variety of commercial products (Dawn™ Hand Dish Wash, Suave® Body Wash, Tide® Liquid Laundry Detergent, and Fabuloso® Cleaner) when 0.5 wt. % aromatic alcohol was incorporated into the products. Foam stability of these commercial products was not reduced by the addition of aromatic alcohol.

These results demonstrate that aromatic alcohols can be used in process to reduce or eliminate foaming without compromising the foam stability of the end use product. This may afford formulators an opportunity to eliminate conventional defoamers in process, particularly those subject to regulatory scrutiny.

### Example 3. Degreasing

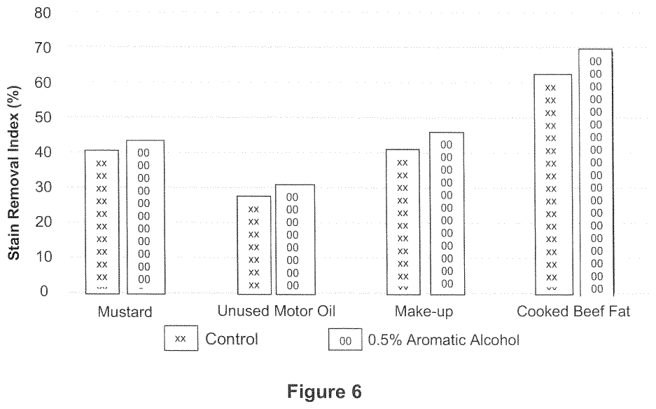

When used in Tide® Free & Gentle™ laundry detergent at 0.5 wt. % based on the weight of the laundry detergent, an aromatic alcohol (R=3, 3-phenylpropanol) exhibited stain removal properties on mustard stains as well as “greasy” type stains including motor oil, make-up, and cooked beef fat. FIG. 6 shows that roughly a 10% increase in Stain Removal Index (%) was achieved over the control (laundry detergent without an aromatic alcohol).

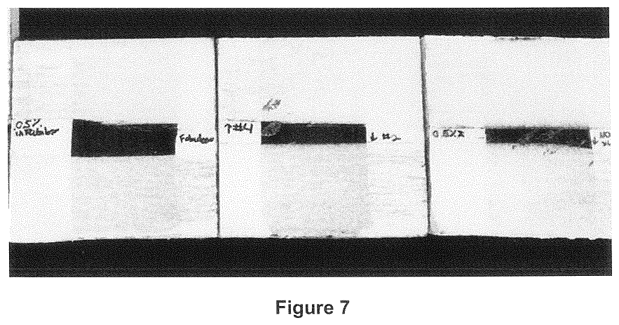

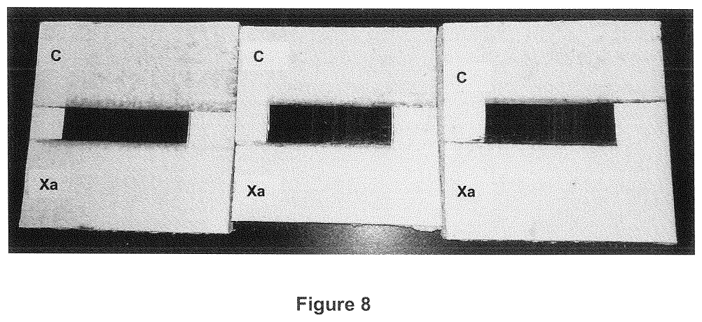

Degreasing properties of an all-purpose cleaner were also improved with the addition of aromatic alcohols. Painted wallboard tiles stained with greasy soil were cleaned using Fabuloso® with and without the addition of 0.5 wt. % aromatic alcohol (R=3, 3-phenylpropanol). The addition of the aromatic alcohol resulted in a 7% increase in greasy soil removal, from 89% to 96% compared to the control, as determined by reflectance values collected by spectrophotometer (FIG. 7). FIG. 7 shows that the portion of tiles (upper) cleaned with Fabuloso® containing 0.5 wt. % aromatic alcohol experienced greater soil removal as compared to the bottom portion of tiles cleaned with Fabuloso® alone. Similar improvement in cleaning was seen with use of 0.5 wt. % aromatic alcohol (R=4, 2-methyl-3-phenylpropanol), with a 5% increase in greasy soil removal (FIG. 8).

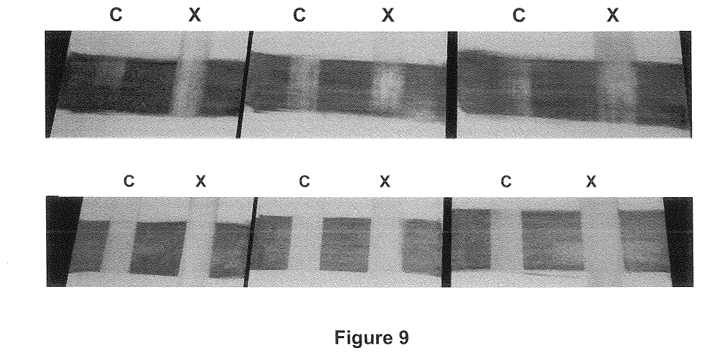

Degreasing properties can be useful in personal care products as well, including without limitation makeup removal products that are micellar waters. Typical micellar water components used in commercial makeup removal include surfactants, humectants, emulsifiers, fragrances, preservatives and pH adjusters. One commercial micellar water (Garnier SkinActive®) used for makeup removal demonstrated increased cleansing ability on long-lasting makeup when 1.0 wt. % aromatic alcohol (R=3, 3-phenylpropanol) was added to the micellar water formulation (FIG. 9). FIG. 9 (top panels across) shows results achieved for Rimmel London® Lasting Finish® lipstick removal. The bottom panels (across) show results achieved for Maybelline® Superstay™ foundation removal. In sets of panels, columns labeled “C” were tested with Garnier SkinActive® micellar water, and columns labeled “X” were tested with Garnier SkinActive® micellar water with 1.0 wt. % aromatic alcohol added. The results demonstrated that the addition of aromatic alcohol improved the degreasing and_cleaning properties of the makeup removal product.

Rimmel London® Lasting Finish® lipstick removal was increased 96% compared to the control (with no aromatic alcohol). For Maybelline® Superstay™ foundation, the addition of 1.0 wt. % aromatic alcohol resulted in a 17% increase in cleaning compared to the control. Cleaning improvement values were determined by spectrophotometer reflectance measurement “L” values, average of three trials, according to the method described above (Equation 1).

### Example 4. Formulation Stability and Cold Processing

Aromatic alcohols can help solubilize hard-to-dissolve formulation ingredients more quickly and/or without the use of heat. The addition of 1.0 wt. % aromatic alcohol decreased the dissolution time of several common, hard-to-dissolve ingredients as shown in Table 1 below. Table 1 reflects the time to completely dissolve ingredients in water (Control) or water with 1 wt. % aromatic alcohol (R=3 denotes 3-phenylpropanol; R=4 denotes 2-methyl-3-phenylpropanol).

TABLE 1

| | Wt. | Time to Dissolve at 22° C. (minutes) | | |
|---|---|---|---|---|
| Ingredient | % | Control | R = 3 | R = 4 |
| Coco fatty alcohol | 2.0 | >15 | 4.0 | 3.5 |
| sulfate | | | | |
| 1,2-octanediol | 1.0 | >30 | 8.1 | 7.8 |

It was also found that the addition of 0.4 wt. % aromatic alcohol (R=2, 2-phenylethanol) solubilized a solution of 2% Glucopon® 600 UP, lauryl/myristyl glucoside surfactant, at room temperature compared to control (results not shown).

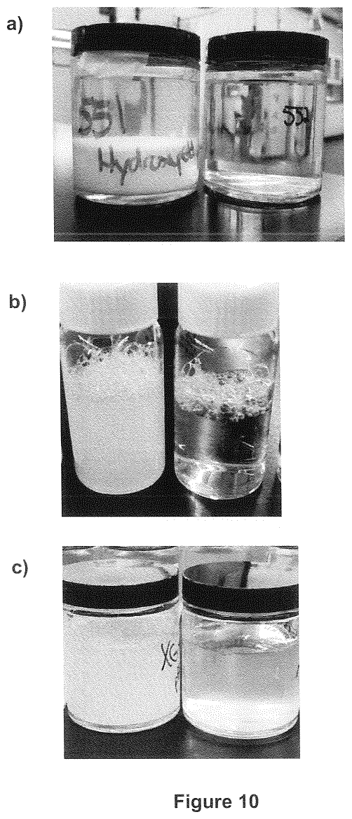

Rapid and complete dissolution of raw materials into aqueous solutions can prevent problems with solution instability over time. For example, a general purpose cleaner containing sodium coco fatty alcohol sulfate and 0.1 wt. % of either hydroxyethyl cellulose or xanthan gum (Table 2) becomes unstable over time across a range of pH values. However, the addition of 0.2-0.5 wt. % of an aromatic alcohol (R=3, 3-phenylpropanol) was observed to solubilize the formulations (FIG. 10).

FIG. 10, panel (a), shows a general purpose cleaner containing hydroxyethyl cellulose at pH 10 in the absence of aromatic alcohol (left side of panel) compared with the same formulation containing 0.2 wt. % aromatic alcohol (R=3, 3-phenylpropanol) (right side of panel) after sitting undisturbed at room temperature one week after preparation. Panel (b) of FIG. 10 compares a general purpose cleaner containing hydroxyethyl cellulose at pH 7 with (right side of panel) and without (left side of panel) the use of 0.5 wt. % aromatic alcohol, stored at room temperature for one week, then shaken to disperse precipitants. Panel (c) of FIG. 10 compares a general purpose cleaner containing xanthan gum at pH 5 with (right side of panel) and without (left side of panel) the use of 0.5 wt. % aromatic alcohol (right,) stored at room temperature for one week, then shaken to disperse precipitants. These observations demonstrated that the use of aromatic alcohols stabilized formulations over a wide range of pH that contained components that are traditionally unstable over time.

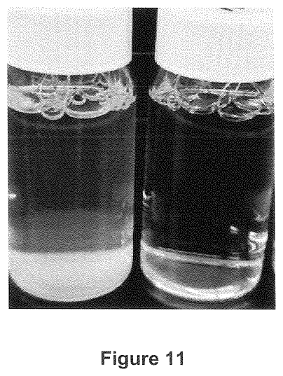

Similar observations were made with formulations of a general purpose cleaner in the absence of hydroxyethyl cellulose or xanthan gum but containing sodium coco fatty alcohol sulfate surfactant (FIG. 11). FIG. 11 compares a sample of a general purpose cleaner at pH 7 with and without 0.5 wt. % aromatic alcohol (R=3, 3-phenylpropanol). The sample on the left (without aromatic alcohol) contained precipitated surfactant after 7 days of aging at 22° C. as compared to the sample on the right containing the aromatic alcohol at 0.5 wt. %.

This solubilizing property can achieve a cost savings for manufacturers due to decreased time and heat requirements during manufacturing. Solutions (such as the general cleaner formula below) that previously required heat to solubilize ingredients can now be processed at room temperature, i.e., cold processed. Formulations traditionally requiring longer mixing times will have that time reduced by first adding an aromatic alcohol to the process.

TABLE 2
Formulation components of a general purpose cleaner.

| Ingredients | Wt. % |
|---|---|
| Water | Q.S. |
| Citric Acid | 1.00% |
| Glycerin | 2.50% |
| Hydroxyethyl Cellulose or Xanthan Gum | 0.10% |
| Sodium Coco Fatty Alcohol Sulfate | 1.30% |
| Sodium Benzoate | 1.00% |
| Sodium Hydroxide | to pH |

### Example 5. Reduction or Elimination of Components of Laundry Detergents Using the Inventive Multifunctional Additives

The use of multifunctional aromatic alcohols for personal care, home care, detergents, industrial, and institutional compositions results in defoaming activity, unexpected stain removal and degreasing, reduction in process aids, and reduction or elimination of fragrances and preservatives. The addition of aromatic alcohols in the manufacturing process reduces the number of processing aids needed, reduces foaming that occurs during the manufacturing process, improves rheology/viscosity profiles resulting less splashing and fewer spills, thus making the process environment safer, and saves energy by allowing for cold processing. The use of aromatic alcohols in laundry detergents obviates the need for enzymes in the removal of greasy stains and improves greasy stain removal without pH constraints typically encountered with detergents using enzyme stain removers. Similarly, the addition of aromatic alcohols in general purpose degreasing cleaners results in greasy stain removal in products with a neutral or acidic pH or without enzymes for fat or grease removal. Those skilled in the art will find other evident advantages for processing and end product properties.

Tables 3-7 below show five typical, commercially available, enzyme free, laundry detergents (Laundry 1, Laundry 2, Laundry 3, Laundry 4 and Laundry 5) with and without the single ingredient, multifunctional additives of the invention. The top sections (“A” portion of the tables) show the standard detergent formulations without the inventive additive and the bottom section (“B” portions of the tables) show the detergents with the additives. The addition of the multifunctional additives of the invention allows a formulator to eliminate a variety of single function components in favor a single multifunctional component, thus reducing the ingredient load of the formulation, while achieving unexpected advantages in processing and end use properties.

TABLE 3 A
Without Additive

| Laundry 1 | | |
|---|---|---|
| Ingredient | Wt. % | Function |
| Water | 71.70% | |
| Linear Alkylbenzene | 7.50% | anionic surfactant |
| Sulfonic Acid | | |
| Alcohol Ethoxylates, 7 EO | 12.00% | nonionic surfactant |
| Sodium Laureth Sulfate | 3.00% | anionic surfactant |
| Propylene glycol | 3.00% | solvent/process aid |
| Sodium borate | 1.00% | builder |
| EDTA | 0.40% | chelator |
| Oleic acid | 1.00% | defoamer |
| Benzisothiazolinone | 100 ppm | Preservative |
| Fragrance | 0.40% | |
| NaOH/Citric Acid | Varies | pH adjustment |

TABLE 3 B
With Additive
Laundry 1: Additive reduces required level of
preservative, omits need for defoamer, reduces PG solvent

| Ingredient | Wt. % | Function |
|---|---|---|
| Water | 72.60% | |
| Linear Alkylbenzene | 7.50% | anionic surfactant |
| Sulfonic Acid | | |
| Alcohol Ethoxylates, 7 EO | 12.00% | nonionic surfactant |
| Sodium Laureth Sulfate | 3.00% | anionic surfactant |
| Propylene glycol | 3.00% | solvent/process aid |
| Sodium borate | 1.00% | builder |
| EDTA | 0.40% | chelator |
| 3-phenylpropanol | 0.50% | Multifunctional/ |
| | | defoamer |
| Oleic acid | | defoamer |
| Benzisothiazolinone | 45 ppm | Preservative |
| Fragrance | 0.40% | |
| NaOH/Citric Acid | 8 | pH adjustment |

TABLE 4 A
Without Additive

| Laundry 2 | | |
|---|---|---|
| Ingredient | Wt. % | Function |
| Water | 72.69% | |
| Sodium Citrate | 3.00% | builder |
| Alcohol Ethoxylates, | 10.00% | nonionic surfactant |
| 7 EO | | |
| Sodium lauryl sulfate | 10.00% | anionic surfactant |
| Glycerin | 3.00% | solvent/process aid |
| Tetrasodium glutamate | 0.71% | chelating agent |
| diacetate | | |
| Oleic Acid | 1.00% | defoamer |
| Fragrance | 0.60% | |
| Sodium Benzoate | 2.00% | Preservative |
| NaOH/citric acid | pH 6.8 | |

TABLE 4 B
With Additive
Laundry 2: Additive reduces process aids, preservatives,
omits need for defoamer, enhances greasy stain
removal in absence of enzymes

| Ingredient | Wt. % | Function |
|---|---|---|
| Water | 75.44% | |
| Sodium Citrate | 3.00% | builder |
| Alcohol Ethoxylates, 7 EO | 10.00% | nonionic surfactant |
| Sodium lauryl sulfate | 10.00% | anionic surfactant |
| Glycerin | 1.00% | solvent/process aid |
| Tetrasodium glutamate | 0.71% | chelating agent |
| diacetate | | |
| Oleic Acid | | defoamer |
| 3-phenylpropanol | 1.00% | Preservative/ |
| | | defoamer |
| Fragrance | 0.60% | |
| Sodium Benzoate | 1.25% | Preservative |
| NaOH/citric acid | pH 6.8 | |

TABLE 5 A
Without Additive

| Laundry 3 | | |
|---|---|---|
| | Active | |
| Ingredient | Wt. % | Function |
| Water | 76.05% | |
| Trisodiunn citrate | 3.00% | builder |
| Tetrasodium glutamate | 2.00% | chelating agent |
| diacetate | | |
| Ethanol | 3.00% | solvent |
| Linear Alkylbenzene | 5.00% | anionic |
| Sulfonic Acid | | |
| Sodium Laureth Sulfate | 2.00% | anionic |
| Alcohol Ethoxylates, 7 EO | 7.00% | non-ionic |
| Sodium xylene sulfonate | 1.00% | coupling agent |
| Dimethicone | 0.10% | defoamer |
| Fragrance | 0.65% | |
| Optical brightener | 0.20% | |

TABLE 5 B
With Additive
Laundry 3: Additive Reduces solvent,
omits need for fragrance

| Ingredient | Wt. % | Function |
|---|---|---|
| Water | 77.90% | |
| trisodium citrate | 3.00% | builder |
| tetrasodium glutamate | 2.00% | chelating agent |
| diacetate | | |
| ethanol | 2.00% | solvent |
| Linear Alkylbenzene | 5.00% | anionic |
| Sulfonic Acid | | |
| Sodium Laureth Sulfate | 2.00% | anionic |
| Alcohol Ethoxylates, 7 EO | 7.00% | non-ionic |
| Sodium xylene sulfonate | 1.00% | coupling agent |
| dimethicone | 0.10% | defoamer |
| Fragrance | | |
| 2-methyl-3-phenylpropanol | 0.70% | |
| Optical brightener | 0.20% | |

TABLE 6 A
Without Additive

| Laundry 4 - Typical | | |
|---|---|---|
| Formula in Literature | | |
| Ingredient | Wt. % | Function |
| Water | 79.49% | diluent |
| Trisodium ethylenediamine | 2.00% | soil capturing |
| disuccinate | | agent |
| Tetrasodium glutamate | 1.89% | cleaning agent |
| diacetate | | |
| Propylene Glycol | 1.00% | solvent |
| Alkylbenzene sulfonate | 7.00% | anionic surfactant |
| Alcohol ethoxylate | 10.00% | nonionic surfactant |
| Sodium fatty acids | 3.00% | anionic surfactant |
| Polyethylenimine | 1.00% | soil capturing |
| ethoxylate | | polymer |
| Sodium Cumene Sulfonate | 1.50% | emulsifying/ |
| | | dispersing agent |
| Dimethicone | 0.10% | defoamer |
| Fragrance | 0.50% | |
| Ethanol | 2.00% | solvent/diluent |
| Optical brightener | 0.20% | |

TABLE 6 B
With Additive
Laundry 4: Additive omits need for fragrance,
reduce process aids

| Ingredient | Wt. % | Function |
|---|---|---|
| Water | 72.51% | diluent |
| Trisodium ethylenediamine | 2.00% | soil capturing agent |
| disuccinate | | |
| GLDA | 1.89% | cleaning agent |
| Propylene Glycol | 1.00% | solvent |
| Alkylbenzene sulfonate | 7.00% | anionic surfactant |
| Alcohol ethoxylate | 10.00% | nonionic surfactant |
| Sodium fatty acids | 3.00% | anionic surfactant |
| Polyethylenimine ethoxylate | 1.00% | soil capturing polymer |
| Sodium Cumene Sulfonate | 1.50% | emulsifying/ |
| | | dispersing agent |
| Dimethicone | 0.10% | defoamer |
| Fragrance | | |
| Ethanol | 1.00% | solvent/diluent |
| Optical brightener | 0.20% | |
| 3-phenylpropanol | 0.50% | |

TABLE 7 A
Without Additive
Laundry 5

| Ingredient | Wt. % | Function |
|---|---|---|
| Water | 80.70% | |
| Sodium citrate | 2.00% | builder |
| Sodium gluconate | 2.00% | anti-redeposition agent |
| Iminodisuccinic acid | 1.00% | water softener/ |
| tetrasodium salt | | anti-redeposition agent |
| Alcohol ethoxylate | 6.00% | surfactant |
| Fatty alcohol sulfate | 4.00% | |
| Triethanolamine | 3.00% | defoamer, solvent |
| Limonene | 1.00% | degreaser/fragrance |
| CIT/MIT | 10 ppm | |

TABLE 7 B
With Additives
Laundry 5: Two additives omit preservatives,
fragrances, degreasers, defoamers

| Ingredient | Wt. % | Function |
|---|---|---|
| Water | 83.00% | |
| Sodium citrate | 2.00% | builder |
| Sodium gluconate | 2.00% | anti-redeposition agent |
| Iminodisuccinic acid | 1.00% | water softener/ |
| tetrasodium salt | | anti-redeposition agent |
| Alcohol ethoxylate | 6.00% | surfactant |
| Fatty alcohol sulfate | 4.00% | |
| 4-phenylbutanol | 1.00% | degreaser/fragrance |
| Phenylethyl alcohol | 1.00% | defoamer |
| Triethanolamine | | |
| Limonene | | |
| Preservative | | |

The examples above illustrate the unexpected results achieved with the aromatic alcohols of the invention. Advantages associated with the use of the aromatic alcohols of the invention are evident in both process improvements and end product properties. Process advantages include increased viscosity of products that can reduce splashing or spills in process environment, resulting in a cleaner and safer process environment and less equipment maintenance. Addition of aromatic alcohols saves energy, is more “green” or efficient, reduces production time, increases production/throughput, and hence saves costs of manufacturing. Solubilizing properties allow for pre-blend solutions and reduce solids handling, which is often difficult and messy. Production time may be saved by preparing pre-blends and concentrated pre-blended solutions of raw materials.

Use of aromatic alcohols in process reduces inventory of single function components and the number of tanks required in the process. Fewer ingredients in processing also results in fewer ingredients listed on products.

Cold processing or reduced heat in process is beneficial for formulations with heat-sensitive ingredients and volatile materials, including but not limited to: solvents, enzymes, prebiotics, probiotics, polymers, dyes, fragrances, preservatives, and natural extracts. Reduced heat puts less stress on manufacturing equipment and makes process safer.

In laundry detergents, addition of aromatic alcohol eliminates the need for enzymes to remove greasy stains. Thus, aromatic alcohols can be used in enzyme-free (“non-bio”) or lipase-free laundry detergents as a booster to remove greasy stains. Aromatic alcohols can also improve greasy stain removal in laundry detergents without pH constraints (enzyme-containing laundry formulas are pH 6.5-8.5). A typical enzyme-free laundry detergent commonly would include surfactants, builders, bleaching agents, said anti-deposition agents, foam regulators, corrosion inhibitors, brighteners, fragrances, dyes, and dye transfer inhibitors.

In general purpose degreasing cleaners (see Table 2), addition of aromatic alcohols allows for greasy soil removal in products with neutral or acidic pH, or without lipase or other enzymes for fat removal.

In personal care cleansers, addition of aromatic alcohols can enhance cleaning performance while providing an improved consumer experience, i.e., product “richness”, luxurious feel, and improved skin feel.

Other advantages for processing and end product properties will be evident to one skilled in the art based upon the disclosure herein.

In accordance with the patent statutes, the best mode and preferred embodiments have been set forth, the scope of the invention is not limited thereto, but rather by the scope of the attached claims.

What is claimed is:

1. A single ingredient, multifunctional additive for use in a personal care, laundry, detergent, home care, industrial, or institutional composition, consisting of an aromatic alcohol that is 3-phenylpropanol, 2-phenylethanol, 2-methyl-3-phenylpropanol, methyl benzene ethanol, methyl benzene propanol, or 4-phenyl-1-butanol, present in the composition in an amount up to 2 wt. % based upon the total weight of the composition,
wherein the multifunctional additive reduces or eliminates the need for single function ingredients to modify low and high shear rheology of a composition, reduces temperatures and time required to manufacture the composition, reduces or destabilizes foam generated in a process environment, and solubilizes other components of the composition.

2. A laundry detergent comprising a silicone-free defoaming agent consisting of the single-ingredient, multifunctional additive according to claim 1.

3. A stain removal additive for use in laundry detergent consisting of the single-ingredient, multifunctional additive according to claim 1.

4. A booster composition to enhance the degreasing activity of detergents and cleaning compositions consisting of the single-ingredient multifunctional additive according to claim 1.

5. The single-ingredient multifunctional additive according to claim 1, wherein the additive is present in the composition in amounts of between 0.1 and 0.5 wt. %, based on the total weight of the composition.

6. A method to reduce manufacturing temperature and or time to manufacture of a formulation, comprising the addition of from 0.2 to 1.0 wt. % of the single-ingredient, multifunctional additive according to claim 1, based on the total weight of the formulation.

7. A method to reduce or destabilize foam production generated during the manufacture of products, comprising the addition of about 0.2 to 0.5 wt. % of the single-ingredient, multifunctional additive according to claim 1, based upon the total weight of the product.

8. A method to improve stain removal of a laundry detergent, comprising the addition of about 0.5 wt. % of the single-ingredient, multifunctional additive according to claim 1, based on the total weight of the laundry detergent.

9. An enzyme-free laundry detergent having improved stain removal properties, comprising a stain removal ingredient consisting of the single-ingredient, multifunctional additive according to claim 1, present in amounts up to 2 wt. % based upon the weight of the laundry detergent.

10. A silicone-free defoaming detergent additive, consisting of the single-ingredient, multifunctional additive according to claim 1.

11. A method of improving cleansing ability of a makeup removal product, comprising:
adding the single-ingredient, multifunctional additive according to claim 1, to the makeup removal product in amounts up to 1 wt. %, based upon the weight of the makeup removal product.

12. A method of reducing or eliminating the number of single function ingredients used in a personal care, cosmetic, home care, laundry, detergent, industrial or institutional product, comprising the step of adding the single-ingredient, multifunctional additive of claim 1, to the product prior to processing or during processing of the product, wherein the additive is present in amounts up to 2 wt. % of product, and wherein the additive eliminates or reduces the need for defoamers, degreasing components, stain removers, solubilizers, solvents, preservatives, or fragrances.

13. A laundry detergent comprising:
a. a builder;
b. a nonionic or anionic surfactant;
c. a solvent;
d. a chelating agent;
e. pH modifiers;
f. the single ingredient, multifunctional additive of claim 1, present in amounts up to 1 wt. % based on the total weight of the laundry detergent; and
g. water.

14. A single ingredient, multifunctional additive for use in personal care, laundry, detergent, home care, industrial or institutional products consisting of: 3-phenylpropanol, 2-methyl-3-phenylpropanol, methyl benzene ethanol, methyl benzene propanol, and 4-phenyl-1-butanol, wherein the additive reduces or eliminates the need for single function ingredients to modify low and high shear rheology of a product, reduces the temperatures and time required to manufacture the product, reduces or destabilizes foam generated in a process environment, and solubilizes other components of the composition.
